(19) United States
(12) Patent Application Publication
(10) Pub. No.: US 2025/0018080 A1
(43) Pub. Date: Jan. 16, 2025

(54) FRAGRANCE DISPENSER

(71) Applicant: Andrew McCullough, Martinez, CA (US)

(72) Inventor: Andrew McCullough, Martinez, CA (US)

(21) Appl. No.: 18/767,528

(22) Filed: Jul. 9, 2024

Related U.S. Application Data
(60) Provisional application No. 63/526,887, filed on Jul. 14, 2023.

(51) Int. Cl. A61L 9/12; A61L 9/04; F04D 29/38

(52) U.S. Cl. CPC A61L 9/122; A61L 9/044; F04D 29/388; A61L 2209/15

(57) ABSTRACT

Disclosed herein are systems and methods for dispensing fragrances, essential oils and other aromatics through the employment of a ceiling fan, fan blades and other devices. In some embodiments the fan blade may have an interior cavity for disposing essential oils. In other embodiments a fragrance dispenser may be affixed to the fan blade. Motion of the fan blade uniformly dispenses fragrances in the space about the fan blade. The construction and method of operation of the invention, however, together with additional objectives and advantages thereof will be best understood from the following description of specific embodiments when read in connection with the accompanying drawings.

### PRIORITY

This application claims the benefit of co-pending provisional patent application 63/526,887 filed Jul. 14, 2023 by the same inventor which is included by reference as if fully set forth herein.

### BACKGROUND

Essential oils and other fragrances often include oils derived from herbs, flowers, trees, and other plants. Such oils are typically present as tiny droplets between the plant's cells and can be extracted by several method known to those of skill in the art (e.g., steam distilled, enfleurage (i.e., extraction by using fat), maceration, solvent extraction, or mechanical pressing). When these types of oils are exposed to air they tend to evaporate (i.e., a volatile oil). As a result, many essential oils are colorless, but with age they can oxidize and become darker. Essential oils are insoluble in water and are soluble in alcohol, ether, fixed oils (vegetal), and other organic solvents. Typical physical characteristics found in essential oils include boiling points that vary from about 160° to 240° C. and densities ranging from about 0.759 to about 1.096.

Essential oils typically are named by the plant from which the oil is found. For example, rose oil or peppermint oil is derived from rose or peppermint plants, respectively. Non-limiting examples of essential oils that may be used in the context of the present disclosure include sesame oil, macadamia nut oil, tea tree oil, evening primrose oil, Spanish sage oil, Spanish rosemary oil, coriander oil, thyme oil, pimento berries oil, rose oil, anise oil, balsam oil, bergamot oil, rosewood oil, cedar oil, chamomile oil, sage oil, clary sage oil, clove oil, cypress oil, eucalyptus oil, fennel oil, sea fennel oil, frankincense oil, geranium oil, ginger oil, grapefruit oil, jasmine oil, juniper oil, lavender oil, lemon oil, lemongrass oil, lime oil, mandarin oil, marjoram oil, myrrh oil, neroli oil, orange oil, patchouli oil, pepper oil, black pepper oil, petitgrain oil, pine oil, rose otto oil, rosemary oil, sandalwood oil, spearmint oil, spikenard oil, vetiver oil, wintergreen oil, or ylang ylang. Other essential oils known to those of skill in the art are also contemplated as being useful within the context of the present disclosure and embodiments herein.

Conventional aromatic fragrance dispensing devices utilize a single aromatic, which may be provided in the form of an essential oil or a mixture of essential oils. The oil or oil mixture is traditionally stored in a small cavity within the dispensing device. The device may include heating elements contained within a base of the cavity to facilitate the release of the scent by vaporizing the essential oils. The dispensing device is typically activated manually; for example, by using a simple on/off toggle located on the device.

Conventionally, the aromatic dispensing device is limited by being only in a single place. Accordingly, the fragrance will be strong near the dispensing device and fainter as one moves away from the device. This leaves an unpleasant sensation to people in the room because of the aroma variability.

### SUMMARY

Disclosed herein are systems and methods for dispensing fragrances, essential oils and other aromatics through the employment of a ceiling fan, fan blades and other devices. In some embodiments the fan blade may have an interior cavity for disposing essential oils. In other embodiments a fragrance dispenser may be affixed to the fan blade. Motion of the fan blade uniformly dispenses fragrances in the space about the fan blade. The construction and method of operation of the invention, however, together with additional objectives and advantages thereof will be best understood from the following description of specific embodiments when read in connection with the accompanying drawings.

### BRIEF DESCRIPTION OF THE DRAWINGS

FIG. 1 shows a break away illustration of a conventional ceiling fan.

FIG. 2 shows an alternative embodiment of a fragrance device.

FIG. 3 shows a pad that may be employed in some embodiments.

FIG. 4 shows a representative image of a fan blade as may be employed in certain embodiments.

### DESCRIPTION

### Generality of Invention

This application should be read in the most general possible form. This includes, without limitation, the following:

References to specific techniques include alternative and more general techniques, especially when discussing aspects of the invention, or how the invention might be made or used.

References to “preferred” techniques generally mean that the inventor contemplates using those techniques, and thinks they are best for the intended application. This does not exclude other techniques for the invention, and does not mean that those techniques are necessarily essential or would be preferred in all circumstances.

References to contemplated causes and effects for some implementations do not preclude other causes or effects that might occur in other implementations.

References to reasons for using particular techniques do not preclude other reasons or techniques, even if completely contrary, where circumstances would indicate that the stated reasons or techniques are not as applicable.

Furthermore, the invention is in no way limited to the specifics of any particular embodiments and examples disclosed herein. Many other variations are possible which remain within the content, scope and spirit of the invention, and these variations would become clear to those skilled in the art after perusal of this application.

### Lexicography

The terms “effect”, “with the effect of” (and similar terms and phrases) generally indicate any consequence, whether assured, probable, or merely possible, of a stated arrangement, cause, method, or technique, without any implication that an effect or a connection between cause and effect are intentional or purposive.

The term “Internet Protocol Security” (IPsec) generally means a protocol suite for securing Internet Protocol (IP) communications by authenticating and encrypting each IP packet of a communication session.

The term “relatively” (and similar terms and phrases) generally indicates any relationship in which a comparison is possible, including without limitation “relatively less”, “relatively more”, and the like. In the context of the invention, where a measure or value is indicated to have a relationship “relatively”, that relationship need not be precise, need not be well-defined, need not be by comparison with any particular or specific other measure or value. For example and without limitation, in cases in which a measure or value is “relatively increased” or “relatively more”, that comparison need not be with respect to any known measure or value, but might be with respect to a measure or value held by that measurement or value at another place or time.

The term “substantially” (and similar terms and phrases) generally indicates any case or circumstance in which a determination, measure, value, or otherwise, is equal, equivalent, nearly equal, nearly equivalent, or approximately, what the measure or value is recited. The terms “substantially all” and “substantially none” (and similar terms and phrases) generally indicate any case or circumstance in which all but a relatively minor amount or number (for “substantially all”) or none but a relatively minor amount or number (for “substantially none”) have the stated property. The terms “substantial effect” (and similar terms and phrases) generally indicate any case or circumstance in which an effect might be detected or determined.

The terms “this application”, “this description” (and similar terms and phrases) generally indicate any material shown or suggested by any portions of this application, individually or collectively, and include all reasonable conclusions that might be drawn by those skilled in the art when this application is reviewed, even if those conclusions would not have been apparent at the time this application is originally filed.

### DETAILED DESCRIPTION

Specific examples of components and arrangements are described below to simplify the present disclosure. These are, of course, merely examples and are not intended to be limiting. In addition, the present disclosure may repeat reference numerals and/or letters in the various examples. This repetition is for the purpose of simplicity and clarity and does not in itself dictate a relationship between the various embodiments and/or configurations discussed.

### System Elements

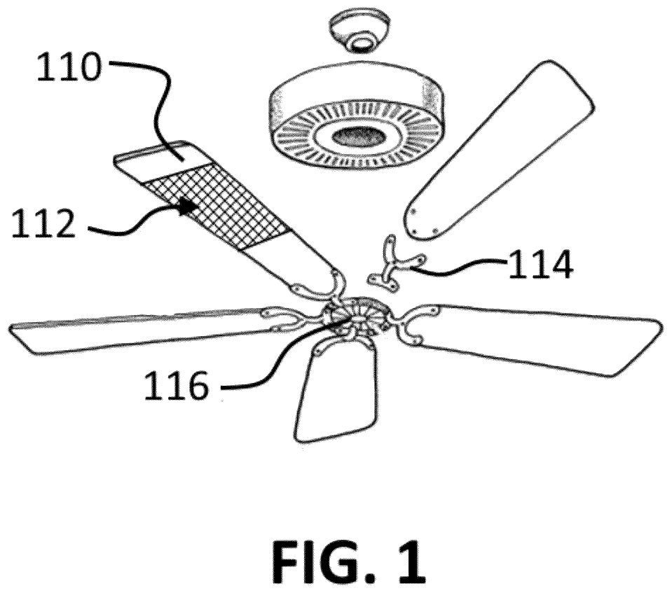

FIG. 1 shows a break away illustration of a conventional ceiling fan. In FIG. 1 a fan blade 110 is attached to the hub 116 of the ceiling fan through an iron 114. Disposed about the fan blade is a flexible fragrance device 112. The fragrance device is secured to the fan blade 110 by wrapping it around the fan blade 110 and securing it in place using VELCO adhesive technology (not shown on this drawing).

The flexible fragrance device 112 may be made from cloth, sponge, paper, and the like. The device may be capable of absorbing a fragrance carrier while maintaining flexibility. Some embodiments may include elastic banding, or elastic material, to secure the device 110 in place. Additional embodiments may be formed using a “sock” type structure that fits over the end of the fan blade 110 to effectuate the same result. The sock-like structure may be closed or open “toe” depending on the a users requirements or desires.

The fragrance device may be impregnated with an aromatic substance. The substate compositions of the present disclosure may also include any one of, any combination of, or all of the following additional ingredients: water, a chelating agent, a moisturizing agent, a preservative, a thickening agent, a silicone containing compound, an essential oil, a structuring agent, a vitamin, a pharmaceutical ingredient, or any combination of such ingredients or mixtures of such ingredients. In certain aspects, the composition can include at least two, three, four, five, six, seven, eight, nine, ten, or all of these additional ingredients identified in the previous sentence. Non-limiting examples of these additional ingredients are identified throughout this specification and are incorporated into this section by reference. The amounts of such ingredients can range from 0.0001% to 99.9% by weight or volume of the composition, or any integer or range in between as disclosed in other sections of this specification, which are incorporated into this paragraph by reference.

Kits that include the compositions of the present disclosure are also contemplated. In certain embodiments, the composition is comprised in a container. The container can be a bottle, dispenser, or package. The container can dispense a pre-determined amount of the composition, for example, and without limitation, onto a flexible fragrance device. In certain aspects, the compositions are dispensed in a spray, dollop, or liquid. The container can include indicia on its surface. The indicia can be a word, an abbreviation, a picture, or a symbol.

In other embodiments a kit may include a flexible device impregnated with or otherwise embodied with the fragrance compositions. This may allow for a disposable fragrance dispensing device to be sold as ready-to-use.

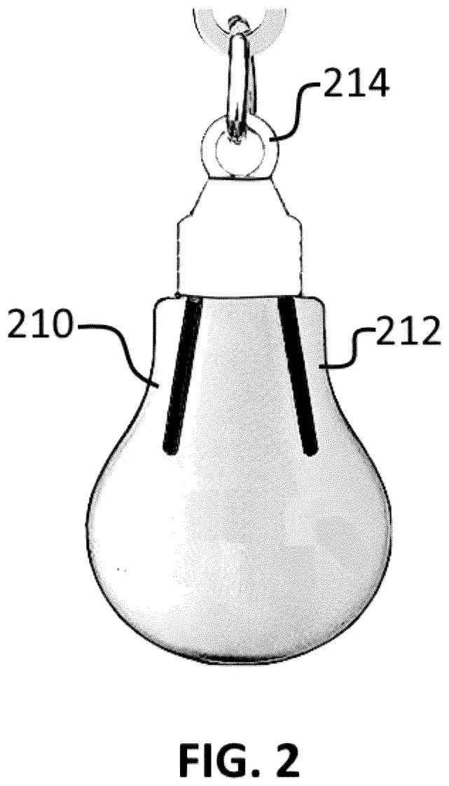

FIG. 2 shows an alternative embodiment of a fragrance device 210. The fragrance device 210 may have openings or slits 212 to expose compositions inside the fragrance device 210 with the outside air. The fragrance device 210 may be made from solid, semi-solid or flexible materials sufficient to contain an aromatic substance within the device 210. The compositions inside the device 210 may be applied to a substance such as a sponge, absorbent paper, gel, liquid, emollient, viscosity control, and the like.

In operation a user may suspend the fragrance device from a ceiling fan. Conventionally, ceiling fans have one or more pull-strings or chains that are used to govern the operation of the ceiling fan. This may include fan speed control and often a lamp affixed to the ceiling fan. Suspending the fragrance device 210 form one or more of these pull-strings would create the desired effect of placing a fragrance dispenser in direct path of air being blown by the ceiling fan. Since ceiling fans usually cover a large area, the aromatic fragrance would be dispensed more uniformly throughout the room or area.

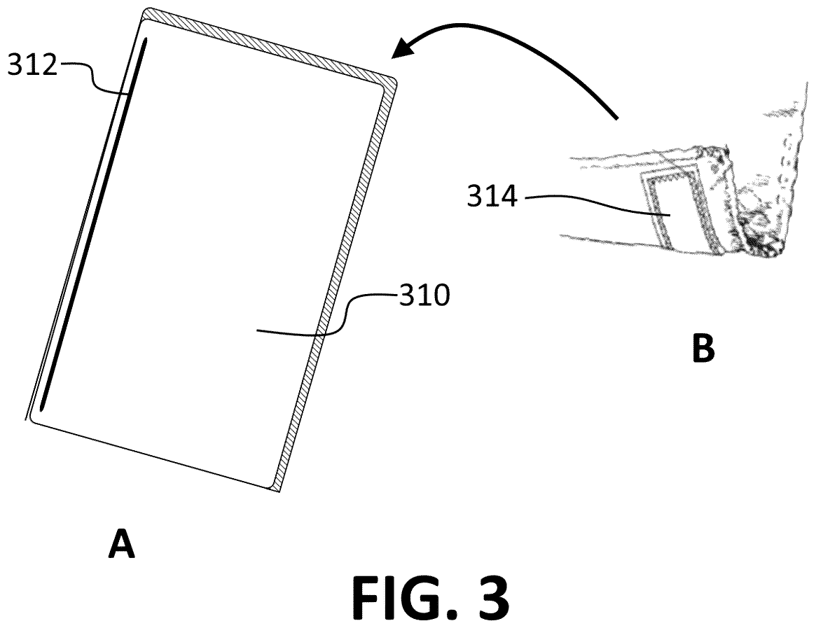

FIG. 3 shows a pad 310 that may be employed in some embodiments. The pad 310 includes a VERCRO portion 312 on one side of the pad 310. While the reverse side includes a mating portion of VELCRO 314. The pad 310 is operable to be wrapped around an object and held in place my mating the two VELCRO portions. In certain embodiments, the pad 310 may be wrapped around the blade of a ceiling fan.

The pad 310 is impregnated with a fragrance substance for dispensing aromatics such as essential oils and deodorizing materials as described herein. The pad 310 may be made from semi-solid or flexible materials sufficient to contain an aromatic substance within the device pad 310 while allowing evaporation to the proximate air. The compositions inside the pad 310 may be applied to a substance such as a sponge, absorbent paper, gel, liquid, emollient, viscosity control, and the like.

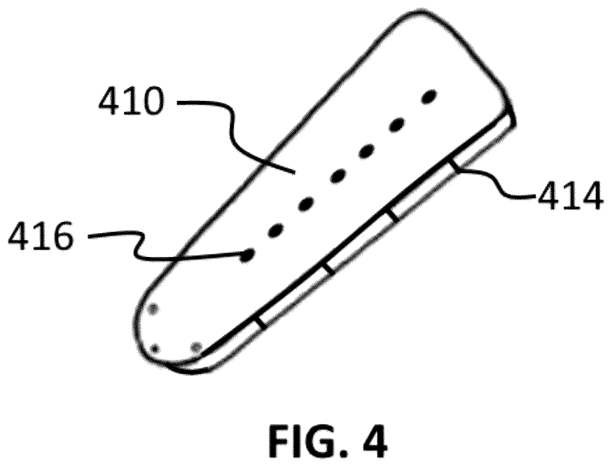

FIG. 4 shows a representative image of a fan blade 410 as may be employed in certain embodiments. The blade 410 has a certain depth and an interior cavity. The cavity provides a space for placement of a fragrance substance as described herein. The blade 410 includes slits or openings 414 that expose the inner cavity to ambient air. The blade 410 may be interchanged with existing blades on a ceiling fan or supplied as part of a new ceiling fan. In operation, the blade 410 spins as part of a ceiling fan, and the substance is exposed to moving air through the slits 414.

In some embodiments, the blade may contain one of more lights such as LEDs 416. These lights may be battery powered or use a kinetic generator which produces power when the fan is in motion. A vibration powered generator is a type of electric generator that converts kinetic energy from vibration into electrical energy. The vibration may be from sound pressure waves or other ambient vibrations. Vibration powered generators usually consist of a resonator which is used to amplify the vibration source, and a transducer mechanism which converts the energy from the vibrations into electrical energy. The transducer usually consists of a magnet and coil or a piezoelectric crystal.

The LEDs 416 are shown in FIG. 4 as a linear array, however, LEDs may be disposed on the blade in any number of patterns and the inventors contemplates LED designs that provide unique light patterns as the fan operates. For example, and without limitation, the LEDs may be disposed on the edge of the blade 410 or disposed to splay light upwards toward a ceiling.

References in the specification to “one embodiment”, “an embodiment”, “an example embodiment”, etc., indicate that the embodiment described may include a particular feature, structure or characteristic, but every embodiment may not necessarily include the particular feature, structure or characteristic. Moreover, such phrases are not necessarily referring to the same embodiment. Further, when a particular feature, structure or characteristic is described in connection with an embodiment, it is submitted that it is within the knowledge of one of ordinary skill in the art to effect such feature, structure or characteristic in connection with other embodiments whether or not explicitly described. Parts of the description are presented using terminology commonly employed by those of ordinary skill in the art to convey the substance of their work to others of ordinary skill in the art.

Certain embodiments may be effectuated using the information in the attached appendix which is incorporated by reference as if fully set forth herein.

The above illustration provides many different embodiments or embodiments for implementing different features of the invention. Specific embodiments of components and processes are described to help clarify the invention. These are, of course, merely embodiments and are not intended to limit the invention from that described in the claims.

Although the invention is illustrated and described herein as embodied in one or more specific examples, it is nevertheless not intended to be limited to the details shown, since various modifications and structural changes may be made therein without departing from the spirit of the invention and within the scope and range of equivalents of the claims. Accordingly, it is appropriate that the appended claims be construed broadly and in a manner consistent with the scope of the invention, as set forth in the following claims.

1. A fan blade including:
a flexible fragrance device, said fragrance device including a portion wrapped around the fan blade,
said fragrance device impregnated with an aromatic substance, said aromatic substance selected from a group including essential oils.

2. The fan blade of claim 1 wherein the aromatic substance is selected from a group including sesame oil, macadamia nut oil, tea tree oil, evening primrose oil, Spanish sage oil, Spanish rosemary oil, coriander oil, thyme oil, pimento berries oil, rose oil, anise oil, balsam oil, bergamot oil, rosewood oil, cedar oil, chamomile oil, sage oil, clary sage oil, clove oil, cypress oil, eucalyptus oil, fennel oil, sea fennel oil, frankincense oil, geranium oil, ginger oil, grapefruit oil, jasmine oil, juniper oil, lavender oil, lemon oil, lemongrass oil, lime oil, mandarin oil, marjoram oil, myrrh oil, neroli oil, orange oil, patchouli oil, pepper oil, black pepper oil, petitgrain oil, pine oil, rose otto oil, rosemary oil, sandalwood oil, spearmint oil, spikenard oil, vetiver oil, wintergreen oil, or ylang ylang.

3. The fan blade of claim 2 wherein the aromatic substance may also include any one of, any combination of, or all of the following additional ingredients: water, a chelating agent, a moisturizing agent, a preservative, a thickening agent, a silicone containing compound, an essential oil, a structuring agent, a vitamin, a pharmaceutical ingredient, or any combination of such ingredients or mixtures of such ingredients.

4. The fan blade of claim 1 wherein the fragrance device includes an elastic portion for securing the fragrance device to the fan blade.

5. The fan blade of claim 1 wherein the fragrance device substantially covers a first end of the fan blade.

6. A fan blade including:
an interior cavity;
an air passage disposed to allow air to pass to the interior cavity;
an aromatic substrate disposed in the interior cavity, said aromatic substance selected from a group including essential oils.

7. The fan blade of claim 6 wherein the aromatic substance is selected from a group including sesame oil, macadamia nut oil, tea tree oil, evening primrose oil, Spanish sage oil, Spanish rosemary oil, coriander oil, thyme oil, pimento berries oil, rose oil, anise oil, balsam oil, bergamot oil, rosewood oil, cedar oil, chamomile oil, sage oil, clary sage oil, clove oil, cypress oil, eucalyptus oil, fennel oil, sea fennel oil, frankincense oil, geranium oil, ginger oil, grapefruit oil, jasmine oil, juniper oil, lavender oil, lemon oil, lemongrass oil, lime oil, mandarin oil, marjoram oil, myrrh oil, neroli oil, orange oil, patchouli oil, pepper oil, black pepper oil, petitgrain oil, pine oil, rose otto oil, rosemary oil, sandalwood oil, spearmint oil, spikenard oil, vetiver oil, wintergreen oil, or ylang ylang.

8. The fan blade of claim 6 wherein the aromatic substance may also include any one of, any combination of, or all of the following additional ingredients: water, a chelating agent, a moisturizing agent, a preservative, a thickening agent, a silicone containing compound, an essential oil, a structuring agent, a vitamin, a pharmaceutical ingredient, or any combination of such ingredients or mixtures of such ingredients.
